(19) United States
(12) Patent Application Publication
(10) Pub. No.: US 2013/0123828 A1
(43) Pub. Date: May 16, 2013

(54) Surgical Retraction Device and Procedure

(71) Applicants: Peter Robert Culmer, Leeds (GB); David George Jayne, Leeds (GB); Anne Neville, Leeds (GB)

(72) Inventors: Peter Robert Culmer, Leeds (GB); David George Jayne, Leeds (GB); Anne Neville, Leeds (GB)

(73) Assignee: THE UNIVERSITY OF LEEDS, Leeds, Yorkshire (GB)

(21) Appl. No.: 13/811,459

(22) PCT Filed: Jul. 25, 2011
(86) PCT No.: PCT/GB11/51415

§ 371 Date: Jan. 22, 2013

(30) Foreign Application Priority Data
Jul. 23, 2010 (GB) 1012336.2

(51) Int. Cl. A61B 1/32

(52) U.S. Cl. CPC A61B 1/32
USPC 606/185

(57) ABSTRACT

The invention provides a retraction device (1) adapted for use in minimally invasive surgery, said device comprising: (a) access means (3); (b) rigid support means; (c) expandable retraction means (2) comprising a multiplicity of radially oriented members (10); (d) flexible attachment means (6); and (e) a multiplicity of magnetic means, wherein said expandable retraction means in its non-expanded form is attached to said rigid support means by said flexible attachment means and is insertable into a subject through said access means, and wherein said multiplicity of magnetic means are comprised in said expandable retraction means. The invention also provides expandable retraction means comprising a multiplicity of radially oriented members and a method for the use of a retraction device in minimally invasive surgery, the method comprising the steps of providing a retraction device according to the first aspect of the invention, inserting said expandable retraction means via said access means through the cavity wall of a subject, deploying the expandable retraction means in its expanded form, magnetising the tissue to be retracted and attaching the magnetised tissue to the retraction device. The device and method according to the invention satisfy the necessary criteria for use in MIS applications and provide for ease in both manufacture and use, and potential application in many areas of MIS.

### FIELD OF THE INVENTION

The invention is concerned with a retraction system for use in surgical procedures which finds particular application in minimally invasive surgery. A method for the use of the system is also described.

### BACKGROUND TO THE INVENTION

The advent of minimally invasive surgery (MIS) has revolutionised clinical practice over the past decade with proven benefits in terms of less postoperative pain, shorter hospital stays, quicker return to normal function, fewer wound complications and improved cosmesis. The continued implementation of this technique has resulted in predictions that 60-90% of all general surgical procedures will be performed using this technology in the future.

However, the application of MIS presents significant challenges due to the fact that the surgeon's hands are replaced by laparoscopic/endoscopic instruments which are manipulated from outside the body cavity and are typically fairly simplistic devices which further limit dextrous or precise movements. Additionally, navigation is often difficult, since the surgeon's view is provided by a 2D assistant-controlled camera.

A key surgical principle, in the context of either open surgery or MIS, is the ability to adequately retract tissues and organs so as to expose the operative field, and to facilitate dissection. This includes the principle of “triangulation” in which opposing forces are applied to open up normal tissue planes.

In MIS, triangulation is made difficult by the restrictive environment of the closed body cavity and the lack of suitable instrumentation. Current retractors for MIS are relatively primitive, consisting of ratcheted devices of varying shapes and sizes. Although some progress has been made in the development of “bending” graspers, significant further advances are necessary in order to improve capability if retraction is to be as good as in open surgery. Most particularly, an improved device/tissue interface is required to reduce the risk of iatrogenic tissue injury, and it is especially desirable that the retraction device should be “hands-free”, thereby enabling a surgeon to concentrate on the operation in progress without dependency on an assistant.

In open surgery, systems which deploy several retractors on a stabilising framework are often used to maximise exposure. An example of such a system is the Omnitract® surgical retractor, wherein a scaffold is anchored to the operating table, and several retracting arms can be attached to the scaffold in order to apply multi-directional retraction to the abdominal wound/tissues. A suitable MIS retraction system might employ a similar strategy.

Various devices are described in the prior art for use in surgical procedures. Thus, for example, US-A-2005/165449 is concerned with a device and system for manipulating a surgical tool at an intended location, e.g., a confined or inaccessible space, which includes a surgical anchor having at least one opening, wherein the opening provides a catch for a pin, and at least one anchor point to position and orient a surgical tool inside a human body. The claimed apparatus and system apparently allows for the use of multiple intra-abdominal surgical tools inserted through a single incision, but is clearly intended for use in standard surgical procedures.

US-A-2010/081876 discloses devices and methods for manipulating scoping devices, surgical tools, and/or tissue. A typical system for manipulating a scoping device includes a scoping device having a working channel, a tether extending through the working channel, and an internal coupling member attached to the tether and positioned adjacent a distal end of the scoping device. The internal coupling member can be magnetically coupled to an external coupling member. Manipulation of a scoping device may be achieved by inserting at least a portion of the device into a body cavity and positioning an external coupling member proximate to an external surface of tissue such that the external coupling member magnetically couples through the tissue to an internal coupling member disposed within the body cavity and attached to a tether passed through at least one working channel of the scoping device. However, this device is unsuited to application in MIS.

Similarly, WO-A-2008/131128 teaches magnetic surgical visualisation and manipulation systems which comprise one or more internal effectors that can be delivered into a body cavity and attached to structures within that body cavity, including tissues, organs, implants, and surgical instruments. At least a portion of each internal effector is responsive to magnetic fields, and can be caused to exert force on and manipulate the structure to which it is attached by an external magnetic manipulator placed outside of the body cavity. Also disclosed are methods of using the surgical visualisation and manipulation systems but, again, these are not appropriate for use in MIS.

US-A-2008/171907, on the other hand, describes methods and devices for grasping and manipulating tissue or organs that are stated to be particularly useful for manipulating tissue or organs during minimally invasive surgical procedures. In a typical embodiment, a magnetic substance is introduced into an organ, and a magnetic element is positioned adjacent the organ to generate a magnetic field between the magnetic element and the magnetic substance, such that the magnetic element is effective to move the organ. However, the document makes no mention of retraction devices for use in MIS.

Clearly, therefore, there is a requirement for a retraction device that offers a solution to the problem of simultaneous multi-directional tissue retraction in MIS, and this is addressed by the presently claimed invention which provides a magnetic retraction device for application in MIS. Specifically, the present inventors have devised an Intra-Abdominal Platform (IAP) which comprises a multi-functional laparoscopic support system adapted to reduce the invasiveness of laparoscopic surgical procedures and facilitate personalised surgery by providing enhanced intra-operative retraction and a stable platform for modular enhancements.

### SUMMARY OF THE INVENTION

Thus, in accordance with a first aspect of the present invention there is provided a retraction device adapted for use in minimally invasive surgery, said device comprising:
(a) access means;
(b) rigid support means;
(c) expandable retraction means comprising a multiplicity of radially oriented members;
(d) flexible attachment means; and
(e) a multiplicity of magnetic means,
   wherein said expandable retraction means in its non-expanded (collapsed) form is attached to said rigid support means by said flexible attachment means and is insertable into a subject through said access means, and wherein said multiplicity of magnetic means are comprised in said expandable retraction means.

In preferred embodiments of the invention, said access means comprises an access port through which a surgical device, such as a standard laparoscopic camera, may be passed. Typically, the access port has a diameter in the region of 5-20 mm, more preferably 10-15 mm and, most preferably, about 10-12 mm. Suitable devices for use as access ports are laparoscopic trocars or ports, which may either be of conventional design or may optionally be of the SILS™ (single-incision) type.

Conventional trocars are well known to those skilled in the art and are shaped like pen and have sharp triangular points. Laparoscopic trocars play an important role in laparoscopic surgery, and instruments such as scissors and graspers may be introduced using surgical trocars. Modern-day trocars have eliminated the need for large abdominal incisions, which has contributed greatly to improved patient care. Surgical trocars are typically single-patient use and disposable and are often entirely without a blade and incision made with a scalpel, or have a linear sharp blade designed to minimize the force required to insert the instrument, making them safer to use when introducing laparoscopic ports and helping to avoid inadvertent tissue damage. Wound closure and healing is much faster as these incisions are smaller and tissue damage to the abdominal wall is decreased.

SILS™ devices are designed to give surgeons the ability to use multiple instruments with maximal manoeuvrability through adjustable cannulas within low-profile malleable ports and typically comprise flexible laparoscopic ports that can accommodate up to three instruments through a single incision.

Typically, said rigid support means comprises an operating table on which a subject is laid, and said expandable retraction means is attached, via said flexible attachment means to said rigid support means.

Said expandable retraction means, or retraction scaffold, comprises a central member and a multiplicity of radially-orientated members which are strategically placed in order to ensure that, in use in their expanded form, they provide the required shape and support to the retractor. Said expandable retraction means is conveniently comprised of a biologically inert polymer.

In preferred embodiments of the invention, said radially-oriented members comprise mechanical members slidably attached to said central axis and interlinked by a series of linking members, typically linking pins, such that they form an umbrella arrangement which, in its expanded form, provides the required shape and support. In alternative embodiments of the invention, said radially-oriented members comprise radially-oriented channels, insufflation of which assists the provision of said shape and support.

Said flexible attachment means preferably comprises a plurality of interlocking rigid members comprising a hinge mechanism located externally to the subject, wherein the hinge mechanism is located adjacent the interlace between said access means and said expandable retraction means, and is adapted so as to ensure that expansion of said retraction means, either mechanically or via insufflation, results in the desired retractor configuration.

Said multiplicity of magnetic means comprises a multiplicity of small magnets which are disposed at positions along the radially-orientated members and around the diameter of the scaffold, thereby providing multiple points for magnetic anchorage. Preferably, said magnets are embedded within the polymer in order to avoid direct contact with tissue.

In certain embodiments, said multiplicity of magnetic means comprises electromagnetic means so that said multiplicity of small magnets comprises a multiplicity of small electromagnets. In said embodiment, the magnetic field can be conveniently controlled, such that its magnitude may be increased or decreased, or it may be switched off completely, according to requirements.

In embodiments of the invention wherein said radially-oriented members comprise radially-oriented channels, said multiplicity of magnetic means may comprise at least one ferrofluid located within said channels.

In further embodiments of the invention, said retraction device additionally comprises a camera, typically a modular camera system.

According to a second aspect of the present invention, there is provided a retraction scaffold which comprises a central member and a multiplicity of radially oriented members.

In certain embodiments of the invention, said central member comprises a central axis to which are slidably attached said multiplicity of radially oriented members, wherein said multiplicity of radially oriented members comprise a multiplicity of mechanical members interlinked by a series of linking members.

Typically, said linking members comprise linking pins and said mechanical members form an umbrella arrangement which, in its expanded form, provides the required shape and support.

In alternative embodiments of the invention, said radially-oriented members comprise radially-oriented channels, insufflation of which assists the provision of said shape and support.

Typically, said retraction scaffold comprises a multiplicity of magnetic means, and said multiplicity of magnetic means are comprised in said radially oriented members.

According to a third aspect of the present invention, there is provided a method for the use of a retraction device in minimally invasive surgery, said method comprising the steps of:
(a) providing a retraction device according to the first aspect of the invention;
(b) inserting said expandable retraction means via said access means through the cavity wall of a subject;
(c) deploying said expandable retraction means in its expanded form;
(d) magnetising the tissue to be retracted; and
(e) attaching said magnetised tissue to said retraction device.

Preferred means for magnetising the tissue to be retracted include the injection of ferrofluids into the tissue or the application of ferrofluid/mucoadhesive to the outer surface of the tissue.

Alternatively, magnetisation of the tissue to be retracted may be achieved by means of magnetic moieties, adapted to be affixed to said tissue. Said magnetic moieties may, for example, be affixed to said tissue by gripping means, such as clips. Thus, by the attachment of a multiplicity of said magnetic moieties to said tissue, said magnetised tissue may then become attached to said retraction device.

### BRIEF DESCRIPTION OF THE DRAWINGS

Embodiments of the prior art and the invention are further described hereinafter with reference to the accompanying drawings, in which:

FIGS. 1(a) and (b) illustrate a typical design of a retraction device according to an embodiment of the first aspect of the invention in use in minimally invasive surgery as applied to a subject, wherein the device comprises mechanical expandable retraction means;

FIGS. 2(a) and (b) depict a retraction device as in FIG. 1 showing the mechanical expandable retraction means in its expanded and collapsed forms;

FIG. 3 shows a retraction device as in FIGS. 1 and 2, including the flexible attachment means, with the mechanical expandable retraction means in expanded form;

FIG. 4 illustrates a retraction device as in FIGS. 1 and 2, including the flexible attachment means, with the mechanical expandable retraction means in collapsed form;

FIG. 5 is a plan view of a retraction device according to the first aspect of the invention comprising mechanical expandable retraction means in expanded form; and

FIG. 6 is a side-elevation of a retraction device according to the first aspect of the invention comprising mechanical expandable retraction means in expanded form.

### DESCRIPTION OF THE INVENTION

A typical device according to a first embodiment of the first aspect of the present invention comprises the following components:
A 10 mm access port comprising a laparoscopic trocar or SILS™ device;
A retraction scaffold made of biologically inert polymer which comprises a multiplicity of radially-orientated members comprised in an umbrella arrangement, expansion of which provides the required shape and support to the retractor;
Attachment means affixed to a rigid support and comprising a hinge mechanism at the scaffold/port interface to ensure that the desired retractor configuration is achieved in its expanded form; and
Small magnets placed at points along the radially-oriented members and around the diameter of the scaffold, and embedded within the polymer so as to avoid direct contact with the tissue, these magnets providing multiple points for magnetic anchorage.

A typical device according to a second embodiment of the first aspect of the present invention comprises the following components:
A 12 mm access port comprising a laparoscopic trocar or SILS™ device;
A retraction scaffold made of biologically inert polymer which contains strategically placed radially-orientated channels, insufflation of which assists the provision of the required shape and support to the retractor;
Attachment means affixed to a rigid support and comprising a hinge mechanism at the scaffold/port interface to ensure that the desired retractor configuration is achieved in its insufflated form; and
Small magnets placed at points along the insufflation channels and around the diameter of the scaffold, and embedded within the polymer so as to avoid direct contact with the tissue, these magnets providing multiple points for magnetic anchorage.

As previously discussed, the prior art surgical retractor devices as used in conventional open surgery, such as the Omnitract®, comprise a scaffold which is anchored to the operating table, and to which several retracting arms are attached in order to apply multi-directional retraction to the abdominal wound/tissues.

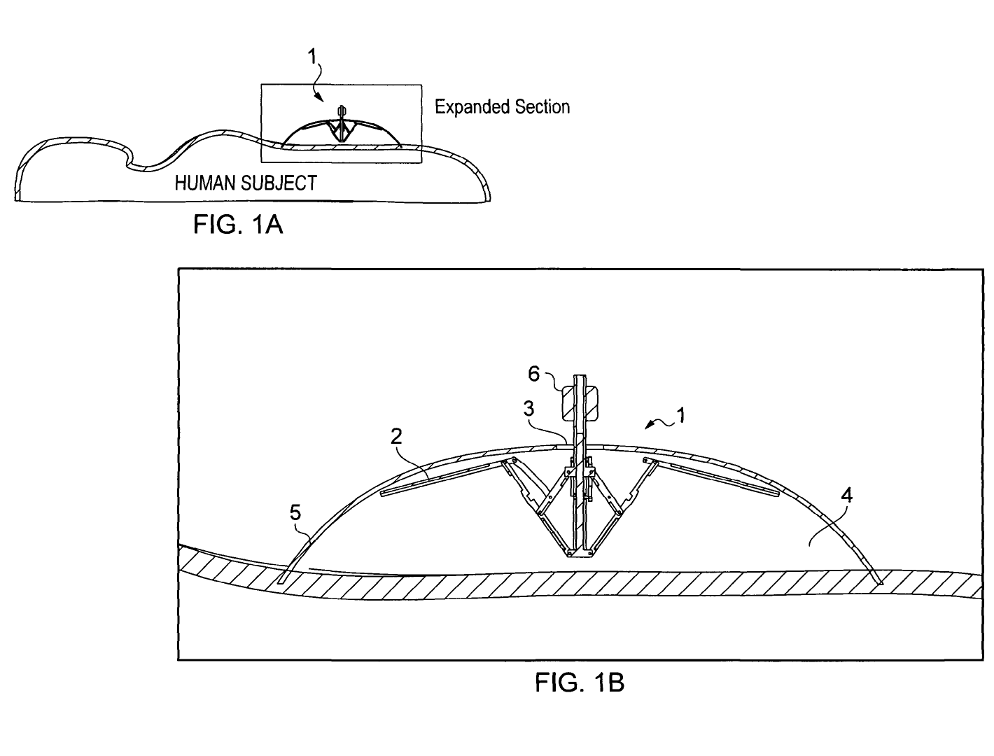

Turning to the diagrams, FIG. 1(a) shows a retraction device according to the present invention applied to a subject. As illustrated in the expanded section shown in FIG. 1(b), the invention provides a retraction device (1) for MIS, the device comprising a retraction scaffold (2) and a standard laparoscopic 10 mm access port (3) through which the retraction scaffold may be passed in its collapsed form into an insufflated cavity (4), for example an abdominal cavity, under the abdominal surface (5) of a subject, thus minimising access trauma. The device is designed to be located at the central laparoscopic access point, which is typically the umbilicus. The device also comprises flexible attachment means comprising external joining mechanism (6) for securing the retraction scaffold to the rigid support means (not shown).

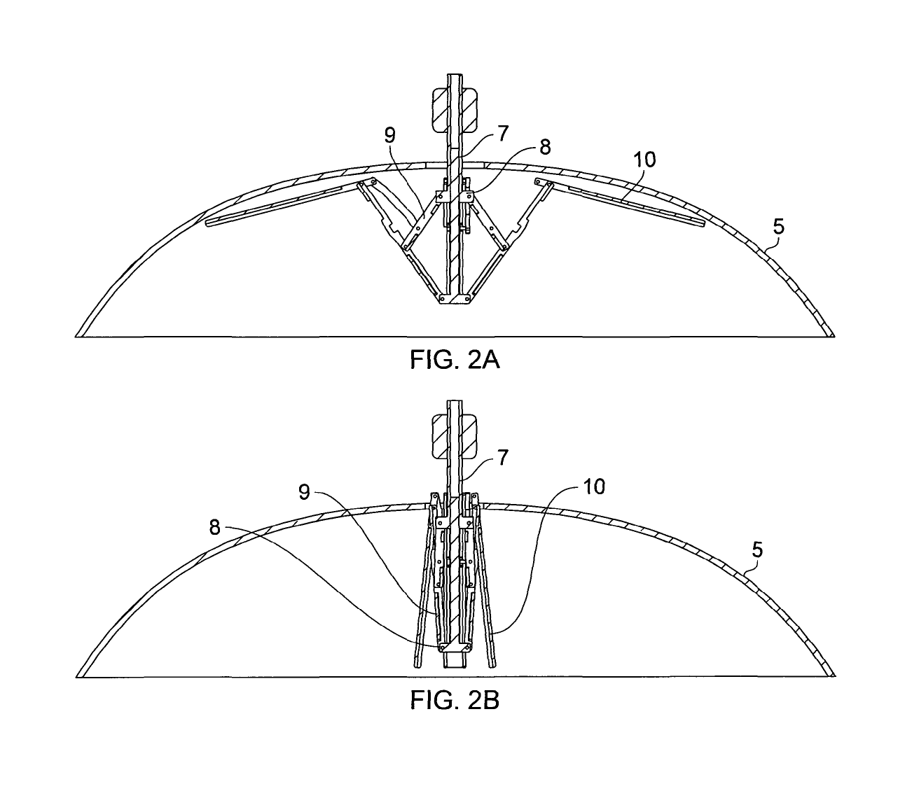

Thus, in operation, deployment is achieved by conforming the retraction scaffold (2) in its collapsed ‘deployment’ state, as shown in FIG. 2(b), wherein its cross-sectional size is minimised, and then inserting the scaffold through the port (3) in the same way as with a conventional laparoscopic instrument. The scaffold structure is independent of the port system, in that it is not permanently fixed thereto, in order to enable a range of different port types to be used. However, the scaffold is fixedly attached to the rigid support means using said flexible attachment means such that it is clamped outside the body on its central shaft to support and stabilise the structure.

Said retraction scaffold (2) is illustrated more clearly in FIGS. 2(a) and 2(b) in its expanded and collapsed forms, respectively. The scaffold comprises a central shaft (7) to which is slidably attached slidably movable member (8), linked to which are, at their first ends, intermediate members (9), said intermediate members being linked at their second ends, by linking members, to folding support members (10), the sections of which are joined together by further linking members. Typical linking members comprise linking pins. Thus, in its collapsed position, the support members (10) are folded such that said retraction scaffold resembles a folded umbrella, as shown in FIG. 2(b), which may conveniently be inserted through the access port.

When deployed in situ, said scaffold is opened to its fully expanded form, as depicted in FIG. 2(a). This is achieved by sliding the slidably movable member (8) along the central shaft (7) until the scaffold is fully expanded, when it is held in the fully expanded form by a ratchet mechanism, as described below. Subsequent release of the scaffold from the expanded form allows it to fold back into its collapsed form, thereby facilitating removal of the scaffold from the subject via the access port.

Typically, said central shaft (7) comprises a hollow member which thereby provides a secondary reduced-diameter access port which facilitates further access to the insufflated cavity via the central access point. In embodiments of the invention, cameras or other instruments may thus be utilised in conjunction with the retraction device of the invention. Generally, the diameter of the reduced diameter port thus provided is in the region of 5-6 mm, typically around 5 mm.

Thus, following deployment, the retraction device is expanded such that the scaffold is arranged as illustrated in FIGS. 1(a) and (b), wherein the structure spans the upper surface of the insufflated abdominal cavity (4). As noted above, this is achieved by sliding the slidably movable member along the central shaft, a process which is effected by means of a ratcheted tensioning system which is actuated externally to the body of the subject via the hollow central shaft. The articulation of the device in this way enables it to conform to the abdominal profile of a range of patient sizes, with the structure then being held in this state by the ratchet mechanism.

As previously noted, fixation of the retraction device (1) relative to the abdominal cavity (4) is achieved using flexible attachment means (6) which typically comprises a rigid support mechanism fixed to the rigid support means, but additionally including a hinge mechanism located adjacent the central access point to facilitate attainment of the desired retractor configuration.

Retraction of tissue and organs is achieved using a system of clips to grasp the desired tissue/organ which clips in turn are anchored onto the IAP framework using mechanical fixings (e.g. hooks) or magnetic elements. After clipping the tissue the surgeon selects an anchor point on the IAP to provide the desired direction and magnitude of traction.

When the scaffold is fully expanded, it is rigid enough to withstand the required retraction forces and, in this position, it is optimally placed to provide maximal retraction of tissue and organs from all quadrants of the cavity whilst remaining out of the field of surgery.

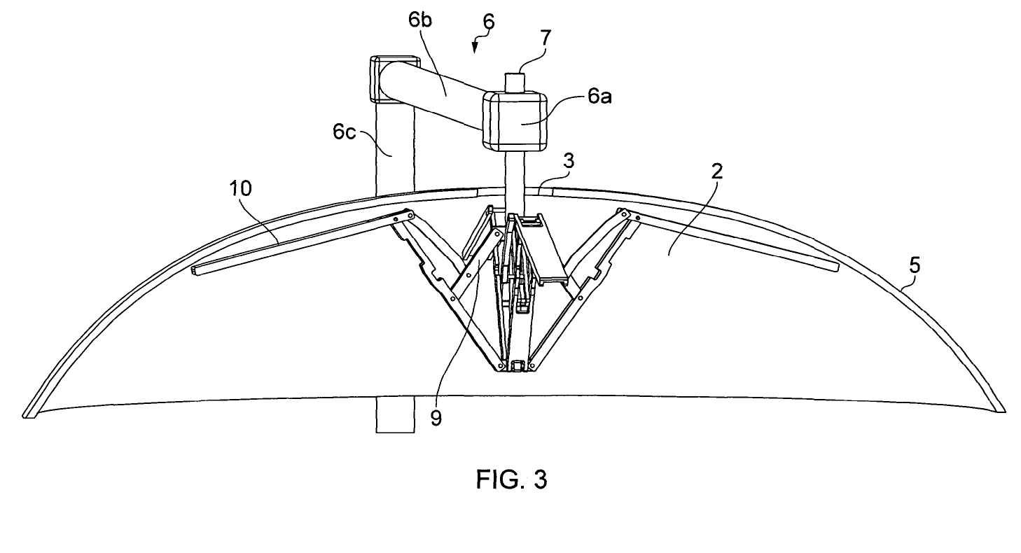

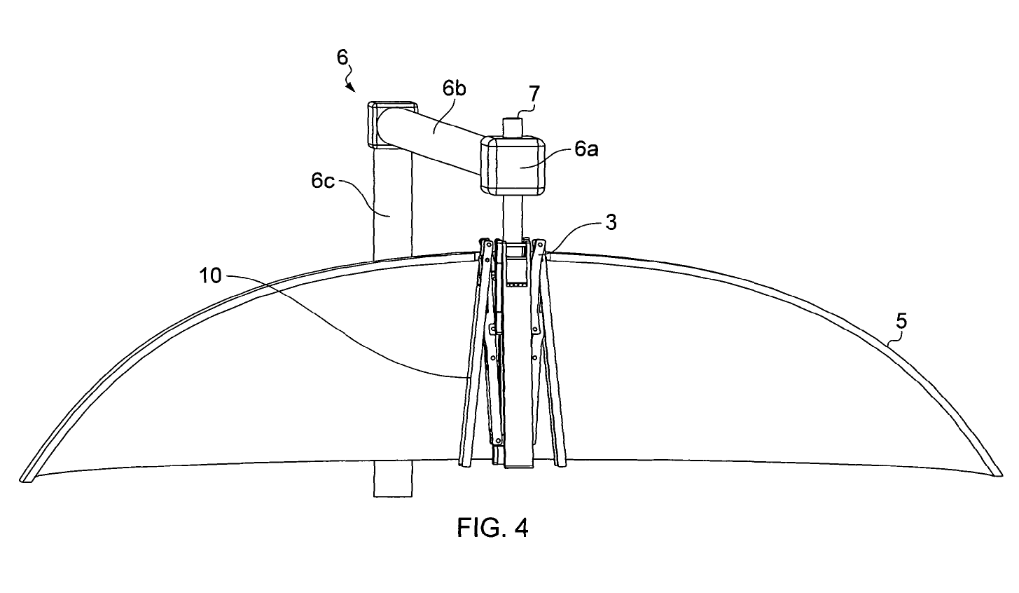

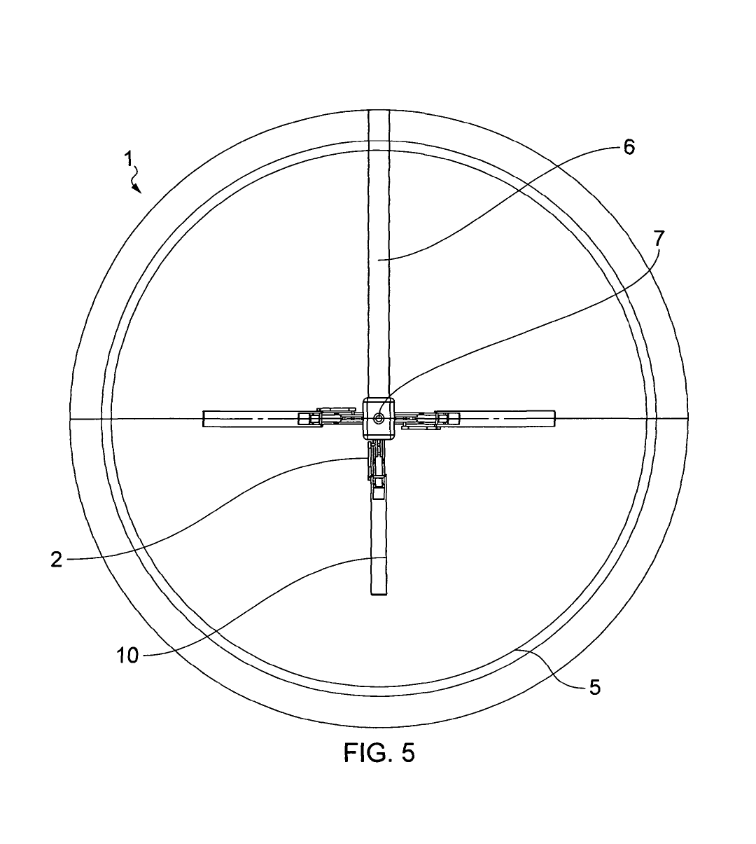

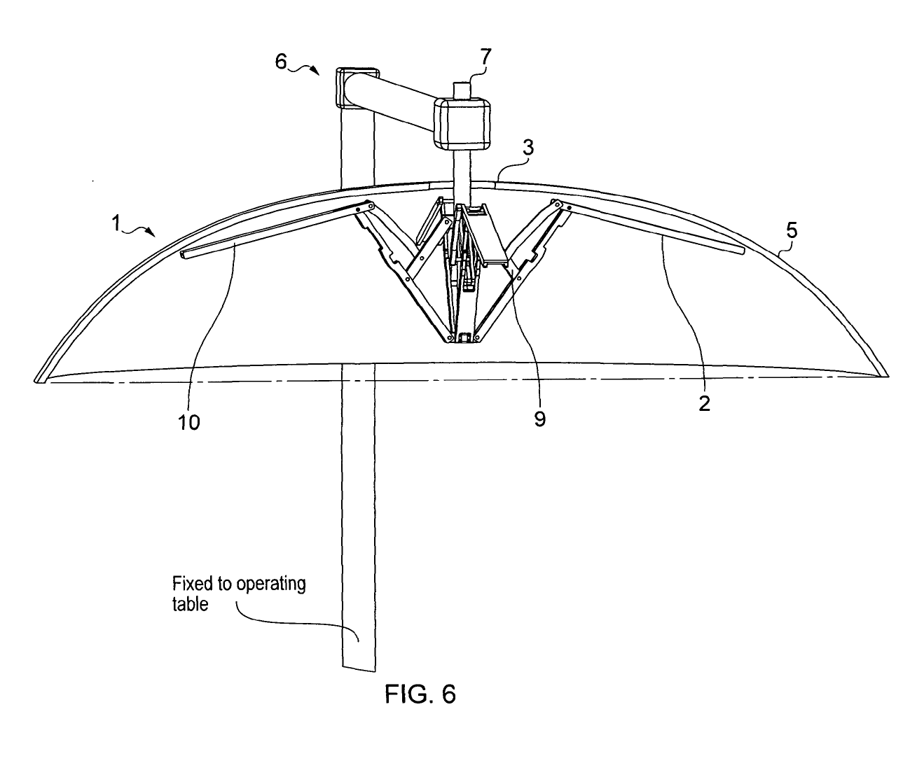

The retraction device of the invention is further illustrated in FIGS. 3, 4, 5 and 6. Thus, in FIG. 3 there is shown a retraction device comprising a retraction scaffold (2) in its expanded form and an access port (3) through abdominal wall (5) through which the retraction scaffold may be passed in its collapsed form. As can be seen from all of FIGS. 3-6, the device also comprises flexible attachment means (6) adapted to secure the central shaft (7) of the retraction scaffold to the rigid support means (not shown); in this embodiment of the invention, flexible attachment means (6) comprises hinge mechanism (6a), horizontal member (6b) and vertical member (6c), wherein members (6b) and (6c) are rigidly connected together. Deployment of the device is achieved by conforming the retraction scaffold (2) in its collapsed state, as shown in FIG. 4, and then inserting it through the port (3).

In certain embodiments of the invention, the hinge mechanism may comprise a ball joint, allowing rotational movement about the joint; preferably, the mechanism additionally comprises gripping means, facilitating the immobilisation of the hinge mechanism once the desired configuration has been attained.

Referring to each of FIGS. 3, 4, 5 and 6, the retraction scaffold is shown to comprise central shaft (7) to which is slidably attached slidably movable member (8), linked to which are, at their first ends, intermediate members (9), said intermediate members being linked at their second ends, by linking members, to folding support members (10), the sections of which are joined together by further linking members. Typical linking members comprise linking pins.

Thus, in its collapsed position, the support members (10) are folded such that said retraction scaffold resembles a folded umbrella, as shown in FIG. 4, which may conveniently be inserted through the access port (3) whilst, when deployed, said scaffold is opened to its expanded form, as depicted in FIGS. 3, 5 and 6. This is achieved by sliding the slidably moveable member (8) along the central shaft (7) until the scaffold is fully expanded, when it is held in the fully expanded form by a ratchet mechanism (not shown). The central shaft (7) comprises a hollow member, providing a secondary reduced-diameter access port.

In a further alternative embodiment of the invention, the device according to the invention incorporates a standard 12 mm access port through which a standard laparoscopic camera can be passed. The access port incorporates the retraction scaffold in its collapsed form. In use, the retraction port is inserted into the body cavity using an open cut-down technique and, once inserted, the retraction element is inflated with carbon dioxide using a laparoscopic insufflator. The design of the retraction scaffold is such that, once inflated, it takes up a position immediately under, and covers most of, the cavity wall. Again, when the scaffold is fully inflated, it is rigid enough to withstand the required tissue retraction forces and, in this position, it is optimally placed to provide maximal retraction to all quadrants of the cavity whilst remaining out of the field of surgery.

When the device of the invention is in use according to the method of the invention, the tissue to be retracted is magnetised, preferably by the injection of ferrofluids into the tissue or the application of ferrofluid/mucoadhesive to the outer surface of the tissue. In this context, the iron-based ferrofluids which are used should be biologically safe, naturally absorbed, readily eliminated from the body, and unlikely to have any unwanted side-effects.

In alternative embodiments, the tissue to be retracted may be magnetised by affixing magnetic moieties, typically comprising clips, to the tissue, thereby facilitating attachment of the thus-magnetised tissue to the retraction device. Following completion of the surgical procedure, the magnetic moieties are removed from the tissue.

Thus, the present invention provides a retraction device that satisfies the necessary criteria for use in MIS applications. Specifically, the device allows several points of device/tissue interaction for multi-directional retraction and maximised retraction forces and facilitates “hands-free” retraction. In addition, it provides reliable and secure retraction forces for a range of tissue types and allows for trauma-free attachment/detachment with no residual tissue injury.

The device is easy to insert and extract, and is of universal application, it is biocompatible with no unwanted local or systemic device interactions, and it can be manufactured as a disposable, but cost-efficient device. Thus, the invention provides a device which is easy to manufacture and use, and which has potential application in many areas of MIS.

Furthermore, in addition to its core retraction functionality, the device according to the invention provides a stable platform for additional, modular enhancements which allow the system to be customised and its functionality extended, for example through computer-assisted imaging and navigation and robotic manipulation. A typical surgical application for the devices and method of the invention is in the field of colorectal resection.

Throughout the description and claims of this specification, the words “comprise” and “contain” and variations of them mean “including but not limited to”, and they are not intended to (and do not) exclude other moieties, additives, components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

The readers attention is directed to all papers and documents which are filed concurrently with or previous to this specification in connection with this application and which are open to public inspection with this specification, and the contents of all such papers and documents are incorporated herein by reference.

1-28. (canceled)

29. A retraction device adapted for use in minimally invasive surgery, said device comprising:
(a) access means;
(b) rigid support means;
(c) expandable retraction means comprising a multiplicity of radially oriented members;
(d) flexible attachment means; and
(e) a multiplicity of magnetic means,
wherein said expandable retraction means in its non-expanded form is attached to said rigid support means by said flexible attachment means and is insertable into a subject through said access means, and wherein said multiplicity of magnetic means are comprised in said expandable retraction means.

30. A retraction device as claimed in claim 29 wherein said access means comprises an access port through which a surgical device may be passed.

31. A retraction device as claimed in claim 30 wherein said access port has a diameter in the region of 10-12 mm.

32. A retraction device as claimed in claim 29 wherein said rigid support means comprises an operating table.

33. A retraction device as claimed in claim 29 wherein said expandable retraction means comprises a multiplicity of radially-orientated members which are adapted such that, in use in their expanded form, they provide the required shape and support to the retractor.

34. A refraction device as claimed in claim 33 wherein said radially-oriented members comprise mechanical members slidably attached to a central axis and interlinked by a series of linking members so as to form an umbrella arrangement which, in its expanded form, provides said shape and support, wherein said linking members optionally comprise linking pins.

35. A refraction device as claimed in claim 33 wherein said radially-oriented members comprise radially-oriented channels, insufflation of which provides said shape and support.

36. A retraction device as claimed in claim 29 wherein said expandable retraction means is comprised of a biologically inert polymer.

37. A retraction device as claimed in claim 29 wherein said flexible attachment means comprises a plurality of interlocking rigid members comprising a hinge mechanism, wherein said hinge mechanism is optionally located adjacent the interface between said access means and said expandable retraction means, and is adapted so as to ensure that expansion of said retraction means, either mechanically or via insufflation, results in the desired retractor configuration.

38. A retraction device as claimed in claim 29 wherein said multiplicity of magnetic means comprises at least one of: (a) a multiplicity of small magnets, (b) electromagnetic means and/or (c) at least one ferrofluid.

39. A retraction device as claimed in claim 38 wherein said multiplicity of small magnets are disposed at positions along the radially-orientated members and around the diameter of the scaffold, thereby providing multiple points for magnetic anchorage.

40. A retraction device as claimed in claim 38 wherein said magnets are embedded within the polymer in order to avoid direct contact with tissue.

41. A retraction device as claimed in claim 34 which comprises the following components:
(a) a 10 mm access port comprising a laparoscopic trocar or SILS™ device;
(b) a retraction scaffold made of biologically inert polymer which comprises a multiplicity of radially-orientated members comprised in an umbrella arrangement, expansion of which provides the required shape and support to the retractor;
(c) attachment means affixed to a rigid support and comprising a hinge mechanism at the scaffold/port interface to ensure that expansion results in the desired retractor configuration; and
(d) small magnets placed at points along the radially-oriented members and around the diameter of the scaffold, and embedded within the polymer so as to avoid direct contact with the tissue, these magnets providing multiple points for magnetic anchorage.

42. A retraction device as claimed in claim 35 which comprises the following components:
(a) a 12 mm access port comprising a laparoscopic trocar or SILS™ device;
(b) a retraction scaffold made of biologically inert polymer which contains strategically placed radially-orientated channels, insufflation of which assists the provision of the required shape and support to the retractor;
(c) attachment means affixed to a rigid support and comprising a hinge mechanism at the scaffold/port interface to ensure that insufflation results in the desired retractor configuration; and
(d) small magnets placed at points along the insufflation channels and around the diameter of the scaffold, and embedded within the polymer so as to avoid direct contact with the tissue, these magnets providing multiple points for magnetic anchorage.

43. A retraction scaffold comprising a central member and a multiplicity of radially oriented members.

44. A refraction scaffold as claimed in claim 43 wherein said central member comprises a central axis to which are slidably attached said multiplicity of radially oriented members, wherein said multiplicity of radially oriented members comprise a multiplicity of mechanical members interlinked by a series of linking members, wherein said linking members optionally comprise linking pins and said mechanical members form an umbrella arrangement which, in its expanded form, provides the required shape and support.

45. A retraction scaffold as claimed in claim 43 wherein said radially-oriented members comprise radially-oriented channels, insufflation of which assists the provision of said shape and support.

46. A retraction scaffold as claimed in claim 43 which comprises a multiplicity of magnetic means, wherein said multiplicity of magnetic means are comprised in said radially oriented members.

47. A method for the use of a retraction device in minimally invasive surgery, said method comprising the steps of:
(a) providing a retraction device as claimed in claim 29;
(b) inserting said expandable retraction means via said access means through the cavity wall of a subject;
(c) deploying said expandable retraction means in its expanded form;
(d) magnetising the tissue to be refracted; and
(e) attaching said magnetised tissue to said retraction device.

48. A method as claimed in claim 47 wherein the means for magnetising the tissue to be retracted comprises (a) the injection of ferrofluids into the tissue, or (b) the application of ferrofluid/mucoadhesive to the outer surface of the tissue or (c) magnetic moieties adapted to be affixed to said tissue, wherein said magnetic moieties are optionally affixed to said tissue by clips.
